(19) United States
(12) Patent Application Publication
(10) Pub. No.: US 2025/0232881 A1
(43) Pub. Date: Jul. 17, 2025

(54) SYSTEM FOR PROCESSING HEALTH INDICATOR VALUES OF A PATIENT

(71) Applicant: LONHEA SA, Fribourg (CH)

(72) Inventor: Michel GOLAY, Founex (CH)

(21) Appl. No.: 18/852,896

(22) PCT Filed: Mar. 30, 2022
(86) PCT No.: PCT/IB2022/052947

§ 371 Date: Sep. 30, 2024

(51) Int. Cl. G16H 50/30; G16H 10/60; G16H 40/67

(52) U.S. Cl. CPC G16H 50/30; G16H 10/60; G16H 40/67

(57) ABSTRACT

A system for processing health indicator values in order to determine a numerical score representative of the general health of a patient, the system including a database that comprises a series of values for indicators of different aspects of health which are calibrated using standardized values. A plurality of input means are each associated with at least one patient. As many intermediate servers are each associated with the input means of one or more patients, and communicate with a master server that comprises a computation unit for all the intermediate servers. Raw data of indicators of different aspects of a patient's health which are received by the input means are calibrated and transmitted to the master server, and the calculated score for which is stored in a display device, for example a smartphone associated with the patient's input means.

The object of the present invention is a system for processing health indicator values of at least one patient to determine for each patient a numerical score representative of the patient's general health in order to help that person regain, optimize and maintain their health and to ensure and increase their well-being and longevity.

The applicant's publication WO2016110734 has already disclosed a method for evaluating a score representing the health of a patient, based on the following considerations.

Immediate access to the results of scientific research has led to a phenomenal acceleration in our understanding of human physiology. Despite this, patients are still too often treated according to erroneous or incomplete concepts. This method is based solely on recent, validated research, in order to offer patients optimal care.

Health is the result of a balance between two parts of the body, the gastrointestinal system or digestive brain, and the nervous system or brain.

The digestive brain must provide energy, essential nutrients, vitamins and trace elements. It must also act as a protective barrier in the same way as the skin, lung mucosa and blood-brain barrier, to prevent the entry of unwanted intruders. It communicates continuously with the brain via the enteric nervous system and numerous hormones, lipoproteins, interleukins and other substances, some of which are still unknown.

The digestive interface needs to be considered in its entirety. This includes the thousands of species of bacteria found in a person's digestive tract. They have lived in symbiosis with generations of individuals for thousands of years. Each individual feeds on the result of their own metabolism. They provide around 50% of each individual's essential nutrient intake. They also produce a lot of lipoproteins, which are able to send information to the brain. They play a fundamental role in immune functions and, above all, in the production of inflammation, which can affect the whole body. The latest scientific evidence clearly demonstrates the links between intestinal flora and certain diseases, such as obesity and type 2 diabetes. Ensuring the quality of an individual's intestinal flora is fundamental to maintaining good health.

The intestinal mucosa acts as a real barrier. It consists of juxtaposed cells held together by tight junctions. The integrity of these links is essential. The intestinal mucosa is the boundary between the outside and inside of the body. So it's not surprising that more than half of an individual's immune defenses are located along the digestive tract. When a foreign bacterium or protein penetrates this protective layer, the immune system must take over in order to rid the individual of it. This can mean considerable energy expenditure for the body. That energy will no longer be available for other functions. It is therefore vital to ensure that the intestinal cells (enterocytes) receive optimum nutrition and that nothing attacks the tight junctions.

The brain is the driver, regulator, and coordinator of all essential functions. For this brain to function properly, it needs to be well protected by the digestive brain and constantly activated. Physical activity, movement via stimulation of cerebellar neurons, is the most important activation. Other stimuli, visual, auditory, gustatory and tactile also play this role.

Over the course of evolution, the brain has changed. Different zones can be distinguished depending on the stage of development. The primitive brain, known as the nervous or reptilian system, is the most important area when it comes to health. It manages essential functions and ensures proper coordination of the various cycles that punctuate an individual's day. The other zones have regulatory actions on the reptilian brain.

Evaluating the functioning of all these zones is therefore vital if the individual is to know the state of their of health. This has been enabled by new discoveries in the field of functional neurology. It is therefore possible to stimulate or inhibit certain brain areas in order to modify their regulatory effects on the nervous system.

In addition, the marker of cellular distress is well known. It is oxidative stress.

What's more, doctors and patients alike are less and less available, and while finding an appointment in the event of illness is still manageable, scheduling appointments for medical follow-up is increasingly complicated.

Today, people have applications on their smartphones, for example, to track their sporting performance or their night's sleep, as well as a medical record held by every doctor they visit.

The goal of the present invention is a system for processing health indicator values of at least one patient to determine for each patient a numerical score representative of the patient's general health in order to help that person regain, optimize and maintain their health and to ensure and increase their well-being and longevity.

The invention relates to a system for processing health indicator values of a patient to determine for each individual patient, among a plurality of patients, a numerical score representative of the patient's general health. The system comprises a database comprising a series of indicator values of different health aspects calibrated using standardized values, means for inputting a series of health indicator data of a patient among said standardized values, a unit for storing data received by the input means, and a unit for computing a numerical score representative of the general health of a patient according to a weighting of the health indicator data values of a patient.

Such a system derives from the implementation of the method described in WO2016110734 through a generalization of the health indicator values and their groupings as well as the weighting applied, whereas the method of WO2016110734 which will be described later involves defined health indicator values and their groupings and a particular weighting.

One aim of the present invention is to provide such a system which can implement the method of WO2016110734 in an easier way to compute the health score and in particular by making the score more quickly accessible to each patient, which is particularly advantageous when the system treats a large number of patients.

A further aim of the invention is to provide such a system that can implement other methods with other health indicator values and according to other weightings of a patient's health indicator values.

According to the invention, the system comprises: a plurality of input means, each of which is associated with at least one patient; a plurality of intermediate servers, each intermediate server being associated with the input means of one or more patients; and a master server for all the intermediate servers, the master server comprising the computing unit. An electronic sensor device comprises at least one sensor associated with input means for receiving, from these input means, raw data indicators of different aspects of a patient's health. Calibration means, integrated into the input means, intermediate servers, or master server, are arranged to calibrate the raw sensor data according to the calibrated and standardized values from the database and to transfer the calibrated values to the storage unit which is arranged to transmit said calibrated data to the computation unit of the master server.

Each intermediate server forms a platform with the master server for exchanging health score data computed by the master server's computation unit. This data exchange platform is arranged to store each patient's general health score in a display device associated with said patient-specific input means. The master server's computation unit is arranged to compute a patient's health score instantaneously each time the input means integrate patient health indicator data whose value affects the computation of said health score, so as to automatically update the general health score of each patient stored in the display device.

WO2016110734 to the applicant defines its method for determining a numerical score representative of a patient's general health based on a series of indicators related to the patient's general state of health and stored in a database, which method can be implemented by the system of the present invention. This method comprises the following steps:
in an initial calibration step, a database is established from a series of indicators relating to the general state of the patient's health, including indicators relating to oxidative stress, indicators relating to the functions of the digestive brain, indicators relating to the functions of the reptilian brain and indicators relating to the patient's physical abilities, coupled with information on the general state of their health, each individual indicator being assigned a numerical value between 0 and 10, and then
a statistical analysis of this database is performed in order to
to establish a score for each of said patient indicators, ranging from 0 to 10, as a function of a measured value of the patient's health status indicators in relation to reference values,
to establish four groups made up of the said indicators stored in the database, i.e.:
a group of oxidative stress indicators, hereafter Group 1;
a group of indicators relating to the functions of the digestive brain, hereinafter referred to as Group 2;
a group of indicators relating to the functions of the reptilian brain, hereinafter referred to as Group 3; and
a group of indicators relating to the patient's physical abilities coupled with information on the general state of their health, hereinafter Group 4, and
for the patient, a value is assigned to each group between 0 and 100, depending on the patient's indicator scores, the value of each group being determined by the relationship 10*(the sum of the scores from 0 to 10 of each of a number N of indicators in the indicator group taken into consideration for computing the value of each group)/N,
then, on a scale between 0 and 100, a health score S specific to the patient's general health is computed using the formula:

$$S = ( \sum 1 * ( Group 1 Value ) + 2 * ( Group 2 Value ) + 2 * ( Group 3 Value ) + 3 * ( Group 4 value ) ) / 8.$$

The numerical score is determined on a scale from 0 to 100. The value of each group also varies from 0 to 100, while the value of each indicator varies from 0 to 10.

In a preferred embodiment of this method, the Group 1 value is determined using scores related to a digestive analysis, an oxidized LDL (Low Density Lipoprotein) value, a HOMA (homoestasis model assessment) index, a Selenium value, a Ferritin value, and a Zinc value. Group 1 gives a value to oxidative stress.

Oxidative stress is at the root of many chronic diseases, and is the result of highly reactive molecules linked to oxygen, constantly damaging our most vital molecules and ultimately making us ill. The paradox is that these highly reactive molecules, known as free radicals, have important biological functions, notably in cell signaling, and are useful for the individual. In excess, they can also attack all the constituents of living organisms and promote chronic diseases such as cataracts, cancer, coronary heart disease, diabetes, kidney failure, Alzheimer's, Parkinson's, and others. In theory, these free radicals are neutralized or taken care of by the body's own protective mechanisms called antioxidants. Today, antioxidants are emerging as the keys to longevity and our allies in the fight against modern diseases. These are protective elements that intervene by scavenging free radicals. Oxidative stress can lead to physical or nervous fatigue, whether temporary or persistent, as well as eating disorders.

In one embodiment of the method, the Group 2 value is determined using scores related to a digestive analysis, a CRP (C-reactive protein) value, a HOMA index and an Omega 6/Omega 3 ratio value. Group 2 gives a value for digestive inflammation.

In one embodiment of the method, the Group 3 value is determined using scores linked to a health questionnaire, a value linked to the measurement of perception time, a homocysteine value, an Omega 3 index value, a score linked to cardiac variability parameters during the night in particular, a value for the root-mean-square of successive differences in heart rate, a value for the LF/HF (low frequency/high frequency) ratio reflecting overall sympathicovagal balance, a value for the restorative sleep index, and a value linked to measurements taken on the total activity of the autonomic nervous system. Group 3 gives a value for brain function.

In particular, nervous system activity is measured through cardiac variability during exercise and over 24 hours, making it possible to measure sleep quality and individual capacity for recovery. This measure is essential for patient follow-up. The further into the program one goes, the greater the need for improvement.

In one embodiment of the method, the value of Group 4 is determined in particular by means of scores linked to a health questionnaire, the results of which are analyzed and weighted, to an exercise test in terms of speed or power, to the measurement of cardiac amplitude, a score linked to a speed test and a root-mean-square value of successive heart rate differences equal to or less than 10, a somesthesia test, a visual test, a vestibular test, a ferritin value, an HOMA index, a zinc value, a selenium value, a CRP value and an immunity questionnaire value. Group 4 gives a value for general health. This is because a complete check-up, based on a thorough functional neurological examination, makes it possible to determine the level of function of the nervous system, as well as the control capacity of the higher cortical areas. The individual functioning and interaction of the three main sensory systems peripheral, vestibular and ocular are also tested. We also assess motor system adaptation, visual abilities when the head is in motion, and functional abilities of the lower limb joints. Each indicator has a value between 0 and 10.

For example, the Group 1 value can be determined using the following formula: Group 1 value=Σ(((3*Digestive analysis value)+(3*Oxidized LDL value)+2*(Zinc value+Selenium value)+HOMA index value+Ferritin value)*10)/12.

The Group 2 value can be determined using the following formula: Group 2 value=Σ(((5*CRP value)+(3*Digestive analysis value)+2*(Omega6/Omega3 value)+HOMA index value)*10)/1−1.

The Group 3 value can be determined using the following formula: Group 3 value=Σ(((3*Health questionnaire value)+(4*Perception time measurement value)+(5*Autonomous nervous system measurement value)+(3*Cardiac variability parameters overnight value HF)+(3*Cardiac variability parameters overnight value LF)+(3*Homocysteine root-mean-square IF)+(3*Root mean square value of successive heart rate differences)+(2*Homocysteine value)+(LF/HF ratio value)+Omega 3 index value+Sleep recovery index value)*10)/26.

Finally, the Group 4 value can be determined using the following formula: Group 4 value=Σ(((3*Health questionnaire value)+(5*Speed effort test value)+(5*Power effort test value)+(4*Heart amplitude value)+(4*Speed test value and a root-mean-square value of successive heart rate differences of successive heart rate differences)+(2*Somesthetic test value)+(2*Visual test value)+(3*Vestibular test value)+(2*Ferritin value)+Homa index value+Zinc value+Selenium value+(4*CRP value)+(2*Immunity test value))*10)/39.

In summary, the method of WO2016110734 determines a numerical score representative of a patient's overall health based on a weighting of a patient's health indicator values.

To perform the method described in WO2016110734, the computation unit computes a score stored in a storage unit accessible from the master server, intermediate server, or electronic capture device.

In one embodiment of the invention, each of the input means comprises a keyboard for manual data entry as well as an input for computerized data.

In this embodiment, each intermediate server and the computation unit separately process each patient's data.

Also according to this embodiment, each of said input means comprises a device for storing and displaying each patient's general health score.

In one embodiment, the computation unit is parameterized to compute the health score according to at least three and preferably at least four parameters representing different health indicator values.

According to this embodiment, the computation unit is configured to compute the health score according to weighted values of four groups of parameters, representing different values of health indicators corresponding respectively to oxidative stress indicators, digestive brain functions, reptilian brain functions, and general health status of a patient.

Still according to this embodiment, the computation unit is configured to compute the health score by taking into account the values of cardiac amplitude, perception time, and omega 3 index.

In one embodiment, the electronic sensing device of the input means is a smartphone, a connected watch, and/or a heart rate belt.

In another embodiment, the input means with the electronic sensor device with the intermediate server are grouped together in a single device or are integrated into a plurality of devices grouped together at the same location, normally the doctor's office.

Thus, the system according to the invention will have as many input means, each with an intermediate server, as there are doctors, each doctor looking after one or more patients.

The features of the present invention will become clearer upon reading several embodiments given solely by way of example, which is in no way limiting, with reference to the schematic figure, wherein:

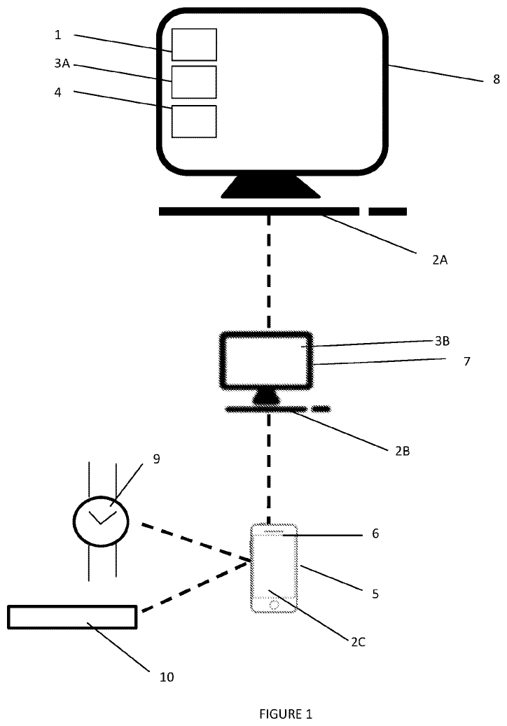

FIG. 1 shows a schematic view of a part of the system illustrating the tracking of a patient through his smartphone, an intermediate server and a master server; and

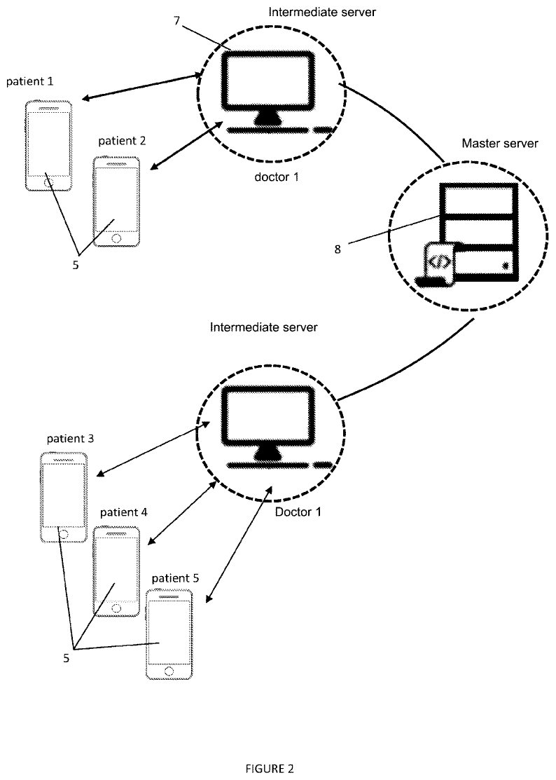

FIG. 2 shows a schematic view of a system illustrating the monitoring of five patients, each via a smartphone, two intermediate servers and a master server;

FIG. 1 shows part of a system for processing a patient's health indicator values to determine a numerical score representative of the patient's general health. The system comprises a database 1 comprising a series of health indicator values, calibrated using standardized values, means for inputting 2A, 2B, 2C a series of health indicator data of a patient among said standardized values, a unit 3A, 3B for storing data received by the input means 2A, 2B, 2C, and a unit 4 for computing a numerical score representative of the general health of a patient according to a weighting of the health indicator data values of a patient.

In this example, the database 1 and storage unit 3A are located on a master server 8, where the computation unit 4 is located.

In this example, the input means 2C comprise a smartphone 5 with a sensor 6 arranged to receive raw health indicator data from the patient and to calibrate this raw data using electronic components according to standardized database values.

In this embodiment, the smartphone 5 is connected to a connected watch 9 and a heart rate belt 10.

The storage unit 3B is integrated in an intermediate server 7, while the storage unit 3A is integrated in the master server 8 and the computation unit 4 is fully integrated in the master server 8.

The database 1 contains, for example, a group of indicators relating to oxidative stress, relating to the functions of the digestive brain, relating to the functions of the reptilian brain, and relating to the patient's physical abilities coupled with information on the general state of their health, quantified as indicated in WO2016110734.

The various input means 2A, 2B, 2C correspond either to the keyboard of the master server 8, the keyboard of the intermediate server 7, or the keyboard of a smartphone 5. These various input means are arranged to enter raw values for all the health indicators according to the groups stored in the database 1. For example, data entered via the smartphone 5 or the intermediate server 7 are calibrated by the master server 8, which instantly returns the health score. In another example, the data captured is automatically calibrated on the smartphone 5 or intermediate server 7 and transferred to the master server 8 to compute a health score.

In the example shown in FIG. 2, each intermediate server 7 is managed by each doctor, who can thus monitor the progress of each of their patients. The intermediate server 7 communicates with the master server 8, which together with each intermediate server 7, constitutes a data exchange platform for health scores computed by the computation unit 4 and for storing the general health score of each in a storage and display means associated with said specific input means for the patient, normally in the intermediate server 7 which transmits the score to the smartphone 5.

The unit 4 for computing a numerical score representative of a patient's general health is arranged to compute a patient's health score instantaneously each time a value of a health indicator affecting said health score is manually or automatically integrated into the data input means 2A, 2B, 2C. In this way, the general health score of each patient, stored on the one hand in the data exchange platform and on the other hand in the storage and display means associated with the specific input means for the patient, is automatically updated.

For example, for each sporting activity, the patient's smartphone 5, connected as shown in FIG. 1 to a connected watch 9 and a heart rate belt 10, records the data affecting the health score, and transmits it to the intermediate server 7, which in turn transmits it to the master server 8. In this way, the patient's health score is automatically updated by the computation unit 4 located in the master server 8.

The computation unit 4 in the master server 8 may for example comprise a computer module programmed to compute a numerical score representative of a patient's overall health according to a weighting of a patient's health indicator values, for example, according to the algorithm described in publication WO201611734.

In the same way, when the doctor enters values that affect a patient's health score on the keyboard 2B of the intermediate server 7, particularly in the context of blood test results, the computation unit 4 in the master server 8 immediately recomputes the patient's health score, and the patient can instantly view their new health score, for example on their smartphone 5.

A phone app enables patients to access their health score and track its progress over time, by day, week or month.

The measured values of the indicators are used to establish a score for each of said indicators, based on the measured value in relation to baseline values.

To perform the method described in WO2016110734, values corresponding to each of the four groups constituted by said indicators are entered into the input means 2A, 2B, 2C, each said group representing information corresponding respectively to oxidative stress, hereinafter group 1; gastrointestinal system functions, hereinafter group 2; nervous system functions, hereinafter group 3; and the patient's physical abilities coupled with information on the general state of their health, hereinafter group 4.

Finally, the computation unit 4, located in the master server 8, uses a computer algorithm to compute a patient-specific health score S, on a scale from 0 to 100, using the formula

$$S = \sum ( Group 1 value + ( 2 * Group 2 value ) + ( 2 * Group 3 value ) + ( 3 Group 4 value ) / ( 2 * Number of groups ) .$$

For example, a patient with a group 1 value of 49.17, a group 2 value of 47.27, a group 3 value of 86.92 and a group 4 value of 99.12 would have a health score S=76.86.

In this example, the closer the health score value is to 100, the healthier the patient.

FIG. 2 shows a system for processing indicator values for the health of five patients to determine a numerical score representative of the general health of each patient. Referring also to FIG. 1, the system comprises a database 1 in the master server 8 and also in the intermediate server 7. This database stores a series of health indicator values calibrated according to standardized values. Each intermediate server 7 is associated with each patient's smartphone 5, constituting means for inputting a series of health indicator data for each patient from among the standardized values. The system further comprises a storage unit 3B in the intermediate server 7 and 3A in the master server 8, for storing data received by input means 2A, 2B, 2C. and a computation unit 4 in the master server 8 for computing a numerical score representative of general health according to a weighting of health indicator values.

In this example, the input means comprise one smartphone 5 for each patient, i.e. five smartphones 5 each comprising a sensor 6 arranged to receive raw health indicator data from each patient and to calibrate this raw data (either in the smartphone 5 or in the intermediate server 7) according to standardized values from the database.

The storage unit is integrated in each smartphone 5, in each of the two intermediate servers 7 and in a master server 8, with the two intermediate servers 7 and the master server 8 receiving data calibrated to standardized values from the database via a means of communication.

In this example, the first intermediate server 7 tracks two patients, and the second intermediate server 7 tracks three patients. In this way, each doctor can monitor the progress of each of their patients. Each intermediate server 7 communicates with the master server 8 constituting a data exchange platform for health scores computed by the computation unit 4 and for storing the general health score of each patient at a location associated with said input means that is specific to the patient, i.e. in smartphones 5.

The unit 4 for computing a numerical score representative of a patient's general health is arranged to compute a patient's health score instantaneously each time that data of a health indicator value affecting said health score is manually or automatically integrated, so that the general health score of each patient, stored in the data exchange platform and in the smartphone 5 constituting a storage/display device associated with said patient-specific input means, is automatically updated by the master server 8.

For example, after each sporting activity, each patient's smartphone 5 records the data affecting the health score, and transmits it to the storage unit 3A, 3B, which transmits it to the master server 8. In this way, the patient's health score is automatically updated by the master server 8.

In the same way, when each of the two doctors enters values affecting the health score on the keyboard 2B of the intermediate server 7, particularly in the context of blood test results, each patient can instantly view their new health score.

For example, a period of four months is sufficient to establish significant changes with a real impact on health. It is only after this period that the improvement in blood balance and sleep quality, the best indicator of the patient's reptilian brain function, will be visible. Comparative analysis of pre- and post-program assessments will demonstrate the improvement in the patient's health score.

1. A system for processing health indicator values to determine for each individual patient, among a plurality of patients, a numerical score representative of the patient's general health, the system comprising:
a database comprising a series of indicator values for different aspects of health, calibrated using standardized values; means for inputting a series of health indicator data for a patient, said data being among said standardized values;
a storage unit for data received by the input means; and
a unit for computing a numerical score representative of a patient's general health according to a weighting of the patient's health indicator data values;
wherein the system comprises:
a plurality of input means, each of which is associated with at least one patient;
a plurality of intermediate servers, each intermediate server being associated with the input means of one or more patients,
a master server for all intermediate servers, the master server comprising the computation unit,
an electronic sensor device comprising at least one sensor associated with input means for receiving, from these input means, raw data indicators of different aspects of a patient's health, and
calibration means, integrated into the input means, intermediate servers, or master server, to calibrate the raw data from the sensor according to the calibrated and standardized values from the database and to transfer the calibrated values to the storage unit which is arranged to transmit said calibrated data to the computation unit of the master server,
wherein:
each intermediate server constitutes with the master server a data exchange platform for health scores computed by the computation unit of the master server, this data exchange platform being arranged to store the general health score of each patient in a display device associated with said input means that is specific to the patient; and
the computation unit of the master server is arranged to compute a patient's health score instantaneously each time the input means, integrate patient health indicator data whose value affects the computation of said health score, so as to automatically update the general health score of each patient stored in the display device associated with said input means that is specific to the patient.

2. The system according to claim 1, wherein at least one of the input means comprises a keyboard for manual data input as well as an input for computerized data.

3. The system according to claim 1, comprising a database in the master server, or a database in each intermediate server.

4. The system according to claim 1, wherein each of said input means comprises a device for storing and displaying each patient's general health score.

5. The system according to claim 1, wherein the computation unit is arranged to compute the health score according to at least three and preferably at least four parameters representing different health indicator values.

6. The system according to claim 5, wherein the computation unit is configured to compute the health score according to weighted values of four groups of parameters, representing different values of health indicators corresponding respectively to oxidative stress indicators, digestive brain functions, reptilian brain functions, and general health status of a patient.

7. The system according to claim 5, wherein the computation unit is configured to compute the health score by taking into account the values of cardiac amplitude, perception time, and omega 3 index.

8. The system according to claim 1, wherein the electronic sensor device of the input means is a smartphone, a connected watch or a heart rate belt.

9. The system according to claim 1, wherein the assembly of each intermediate server with its input means and the electronic sensor device is grouped into a single device or is integrated in several devices grouped at the same location.
